(19) United States
(12) Patent Application Publication
(10) Pub. No.: US 2014/0345629 A1
(43) Pub. Date: Nov. 27, 2014

(54) METHOD AND APPARATUS FOR ASSISTING IN WOUND CLOSURE

(71) Applicant: Rainbow Medical Devices, LLC, Gurnee, IL (US)

(72) Inventor: Dean K. Stockwell, Gurnee, IL (US)

(21) Appl. No.: 13/942,393

(22) Filed: Jul. 15, 2013

Related U.S. Application Data
(63) Continuation-in-part of application No. 29/455,856, filed on May 24, 2013.

(51) Int. Cl. A61B 17/04; A61B 19/00

(52) U.S. Cl. CPC A61B 17/0493; A61B 19/54; A61B 2019/5466; A61B 2019/547
USPC 128/887

(57) ABSTRACT

A device (surgical aid) for assisting wound closure in a surgical procedure setting. A flat plastic sheath (retainer) is inserted into the wound to retain the viscera organs and protect them from cuts or punctures while closing the wound after abdominal surgery or any large body cavity surgery. Just before completion of the wound closure, the device is pulled out of the body via a pull cord and then the wound closure is finished.

### CROSS-REFERENCE TO RELATED APPLICATION

This application is a continuation-in-part of U.S. patent application Ser. No. 29/455,856 filed May 24, 2013, which is incorporated herein by reference in its entirety.

### FIELD OF THE INVENTION

The present invention relates generally to surgical devices to aid with wound closure, and more particularly to devices intended to position or retain internal anatomy and/or protect such anatomy from scrapes, nicks, punctures or cuts as the wound is closed.

### BACKGROUND OF THE INVENTION

During surgical procedures in which a major body cavity has been opened, e.g., the abdomen, chest or any other large body cavity, it is a fundamental requirement to restore the organs in their anatomic position and approximating together the tissue membrane layer at the end of any surgery. For example, in abdominal surgeries, it is necessary to retain the viscera beneath the level of the edges of the peritoneum during the course of parietal closure and protect the patient's anatomy from scrapes, nicks, punctures, tears or cuts while the wound is being closed. This can be difficult to do in some patients and requires care not to injure the anatomy in this way while closing the wound via conventional wound closure means (e.g., suture, medical staple, etc.).

In some instances, a physician may have extra supporting hands from surgical assistants and the like to insure the patient's anatomy remains in its anatomical position and to prevent damaging any of the anatomy as the wound is closed. However, often times there are either not enough hands to perform this task or it is simply determined to be easier to use a medical device to accomplish this task. For example, in cases of emergencies, there may not be adequate enough personnel to assist the surgeon performing the operation.

Several medical devices have been used to help perform this task. For example, in the 1930s, Dr. R. W. McNealy devised a viscera retainer called the “fish” that both retained the viscera and protected the patient's anatomy while the wound was being closed. The product was called the “fish” because its shape was generally that of a sole fish. The product was made of rubber with a metal central stay to reinforce the product. The stay had an eye in its forward end through which braided silk was passed to form a loop. A stainless steel ring was connected to the braided silk loop to form a handle that would remain outside of the wound while the rubber fish body was laid in the wound opening to retain the viscera and protect the patient's anatomy as the wound was closed. When all but a small portion of the wound was closed (e.g., three centimeters), the stainless steel ring would be pulled causing the soft rubber sides of the fish to fold on the central steel reinforcement stay and permit easy removal of the medical device so that the final portion of the wound could be closed. During its day, the advantages of this product were touted as: the ease in sterilizing the product; the fact it could be inserted readily and easily removed; the fact it did not scrape the peritoneal surfaces; and the fact it efficiently retained omentum and viscera during wound closure. One problem with this design, however, was that the rubber body proved too flexible (particularly at the outer perimeter of the fish body) and, thus, did not retain its shape or the viscera and omentum as much as desired.

Several years later, Dr. Jacob A. Glassman improved upon the fish product by adding six ribs extending out to the edges of the rubber fish body from the central reinforcing stay (three on each side of the central reinforcing stay). The ribs were added to add anchorage and retentive power to the fish body, but still allow for the fish to fold into a narrow roll for easy removal when the wound was almost closed. While this product helped the fish retain its shape and the viscera and omentum, the ribs of the fish made it more difficult to remove the device from the patient.

Several third parties have tried to address the problem presented by the ribs extending from the central reinforcement stay. For example, U.S. Pat. No. 4,747,393 (issued to Medwid), U.S. Pat. No. 4,964,417 (issued to Peters), and U.S. Pat. No. 6,736,141 (issued to Freedman) all show similar looking ribless fish-type devices that are meant to retain and protect patient anatomy as discussed above with respect to the McNealy and Glassman devices. Unfortunately, however, the lack of ribs on these devices causes the devices to suffer from the same problems as the original McNealy product or even worse because of the total lack of any reinforcing stay. To date, ribless retainers have been the only solution offered to address the problems that exist with the Glassman retainer despite the many years that have lapsed since this product's initial offering to the public.

Accordingly, it has been determined that a need exists for an improved surgical aid/medical device that helps the physician retain the patient's anatomy and/or protect the patient from scrapes, nicks, punctures, tears or cuts during the wound closure process, but still allows the device to have anchorage and retentive power and yet be extracted or removed easily towards the end of the wound closure process.

### BRIEF DESCRIPTION OF THE DRAWINGS

The invention will be explained in exemplary embodiments with reference to drawings, in which:

FIGS. 1A-E are perspective, top plan, side elevation, front elevation and rear elevation views, respectively, of a surgical device to aid with wound closure in accordance with one exemplary embodiment of the invention disclosed herein (it should be noted that: (a) FIG. 1E is inverted in keeping with the blueprint layout of the drawings; (b) that only one side elevation view is shown in FIG. 1C since the product is symmetrical about its central or longitudinal axis and, thus, the opposite side is simply a mirror image of FIG. 1C; and (c) that the bottom surface of the device is generally flat and unornamented so it has not been shown herein);

FIGS. 2A-D are perspective views of the surgical device of FIGS. 1A-E illustrating a pull cord and handle attached thereto which may be used to remove the surgical device near the end of the wound closure process and showing the device in a wound opening, showing the wound being partially closed over or around the device and showing the device being removed from the wound using the handle and pull cord, respectively (side, end and bottom views are not shown as they are either similar to those illustrated above or unornamented and flat, thereby, not requiring a drawing illustration);

FIGS. 3A-B are perspective and top plan views, respectively, of an alternate exemplary embodiment of a surgical device in accordance with the invention disclosed herein (side, end and bottom views are not shown as they are either similar to those illustrated above or unornamented and flat, thereby, not requiring a drawing illustration); and

FIGS. 4A-B are perspective and side elevation views, respectively, of an alternate exemplary embodiment of a surgical device in accordance with the invention disclosed herein (side, end and bottom views are not shown as they are either the similar to those illustrated above or unornamented and flat, thereby, not requiring a drawing illustration).

While the invention will be described in connection with preferred embodiments, it will be understood that it is not intended to limit the invention to these embodiments. On the contrary, it is intended to cover all alternatives, modifications and equivalents as may be included within the spirit and scope of the invention as defined by the appended claims. Furthermore, skilled artisans will appreciate that elements in the figures are illustrated for simplicity and clarity and have not necessarily been drawn to scale. For example, the dimensions of some of the elements in the figures may be exaggerated relative to other elements to help to improve understanding of the various embodiments of the present invention or to better illustrate surfaces with shading lines, etc. (e.g., the size of the body, spine and/or ribs may be exaggerated with respect to one another or other components in order to make the drawing more clearly show what is being claimed). Also, common but well-understood elements that are useful or necessary in a commercially feasible embodiment are typically not depicted in order to facilitate a less obstructed view of these various embodiments of the present invention.

### DETAILED DESCRIPTION

A surgical aid or medical device for assisting in a wound closure process of a medical procedure is disclosed herein which utilizes several features to improve the usefulness of the aid and its operation. The aid is used to retain and/or protect patient anatomy by inserting the aid into an open wound to hold or retain the anatomy in place and protect the anatomy from scrapes, nicks, punctures, tears or cuts while the wound is being closed (e.g., sutured or stapled shut, etc.) around or over the surgical aid. The aid is moveable between a first or expanded position wherein peripheral portions of the aid are spaced apart from the spine and a second or retracted position wherein the peripheral portions of the aid are collapsed in toward the spine to allow the aid to be removed from the wound near the end of the wound closure process and through a much smaller wound opening than the original wound opening. In one form, the aid includes a flexible body with a spine member aligned along a central axis of the flexible body. The spine member or component is tapered to make the device easier to remove from the wound after a portion of the wound has been closed around or over the surgical aid. In another form, the aid includes a flexible body and spine with ribs extending from the spine toward peripheral portions or regions of the flexible body to assist in retaining the shape of the flexible body so that it can be easily positioned over a patient's anatomy in a wound to retain the anatomy in position and protect the anatomy as the wound is closed. The ribs may be swept back to make the aid easier to be moved into the retracted position when the aid is removed from the wound after a portion of the wound has been closed around or over the surgical aid. The ribs may alternatively or in addition be made to extend out at different angles with respect to one another to allow the ribs to support a larger area of the peripheral portions of the flexible body and still be easily moved into the retracted position when the aid is removed from the wound. The ribs may alternatively or in addition be made longer, particularly with respect to the spine, to allow the ribs to support more of the flexible body yet still be easy to move into the retracted position when the aid is removed from the wound. Any one or more of these features may be implemented into the surgical aid in order to overcome the above-mentioned problems with conventional surgical aids intended to retain and/or protect patient anatomy.

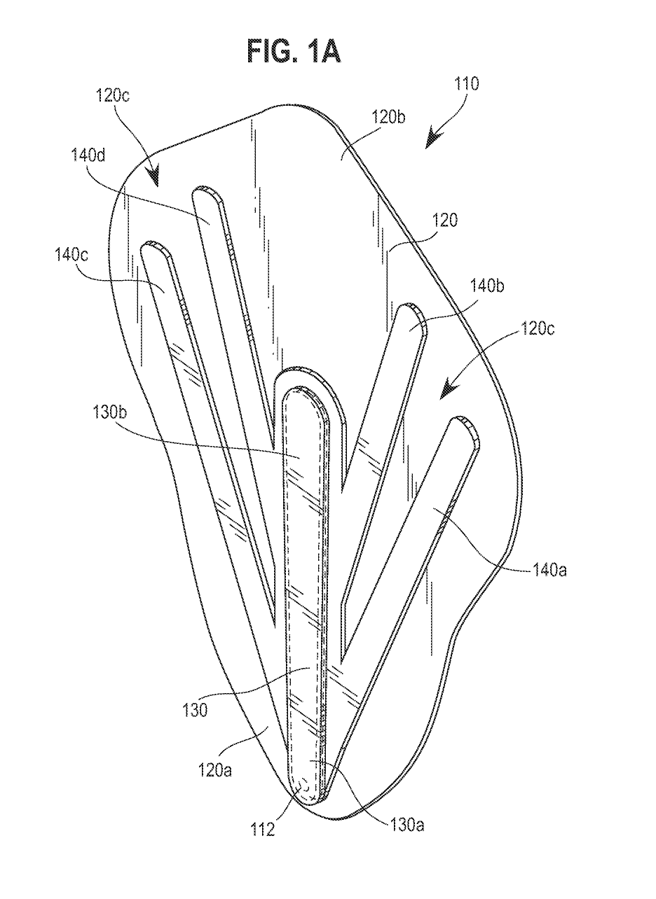

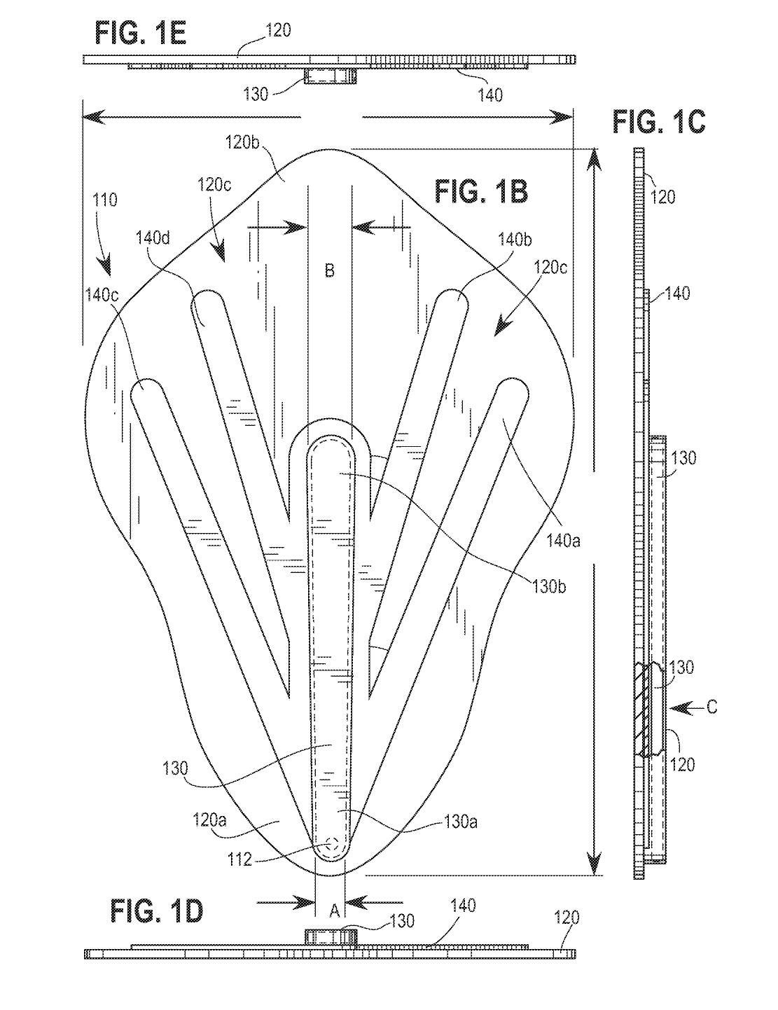

Turning first to FIGS. 1A-E, in which there is illustrated one exemplary embodiment of a surgical aid or medical device 110 in accordance with aspects of the invention, the aid 110 having a body 120 made of a flexible material, such as a flexible plastic or silicon material, and will preferably be a material that is a U.S. Food & Drug Administration (“FDA”) recognized material for medical/surgical applications. For example, in one form the flexible body 120 is made of an FDA grade flexible polyvinyl chloride (“PVC”) material that can be readily sterilized. In other forms, the body 120 may be made of other flexible plastics/silicones with similar flexibility, density, and weight characteristics to the PVC material mentioned for the illustrated embodiment.

In FIGS. 1A-E, body 120 has a first or forward portion 120a and a second or rearward portion 120b and includes a spine member or component 130 which is located along a central axis of the body 120. In the embodiment illustrated, the central axis of the body 120 is coaxial with the longitudinal axis of the aid 110. The body 120 is moveable between an expanded position (see FIGS. 1A-E) wherein peripheral portions of the body 120c are spaced apart from the spine so that the body and spine can be positioned within a wound to retain or protect patient anatomy and a retracted or folded position wherein the peripheral portions of the body are positioned or moved closer to the spine 130 so that the body 120 and spine 130 can be removed from the wound after a portion of the wound has been closed over or around at least a portion of the aid 110.

In the form illustrated, the spine 130 is tapered from a second or rearward portion 130b of the spine toward a first or forward portion 130a of the spine with the forward portion of the spine 130a being positioned at or near the forward portion 120a of the body 120 to further assist in removal of the forward portions 120a, 130a of the body and spine, respectively, from the wound after a portion of the wound has been closed over or around at least a portion of the surgical aid 110. With this configuration, the surgical aid 110 is easier to remove from the partially closed wound because the tapered end 130a of spine 130 and forward end of the body 120a make-up the first portion of the surgical aid 110 that is removed from the patient. The tapering allows this first portion of the surgical aid 110 to be fed through the portion of the wound opening that remains open more easily.

In the embodiment illustrated, the spine 130 is molded from acrylonitrile-butadiene-styrene (“ABS”) or a similar ridged plastic alternative with radiopaque material (e.g., barium sulfate) mixed into the composition and/or applied over the material so that the spine can be seen via medical imaging equipment like electromagnetic radiation imaging equipment (e.g., an X-ray, etc.). Thus, if the surgical aid 110 were ever to be left in a patient at the conclusion of a surgery, this fact could be discovered by taking an X-ray of the patient which would show the presence of the spine 130 of the surgical aid 110. In the form illustrated, the spine 130 will have a height of one hundred thousandths of an inch (0.100″), a length of five and six hundred thousandths inches (5.600″), and taper from a width of six hundred thousandths of an inch (0.600″) at the rear portion 130b thereof (Dimension B in FIG. 1B) to a width of five hundred thousandths inch (0.500″) at the front portion 130a thereof (Dimension A in FIG. 1B). The opening 112 is centered two hundred fifty thousandths of an inch (0.250″) in from the forward edge of the spine 130 and has a diameter of one hundred fifty thousandths of an inch (0.150″). In an alternate form, the spine 130 may be molded from polystyrene with a radiopaque material pigment, filler or coating serving as the contrast medium for x-ray photography.

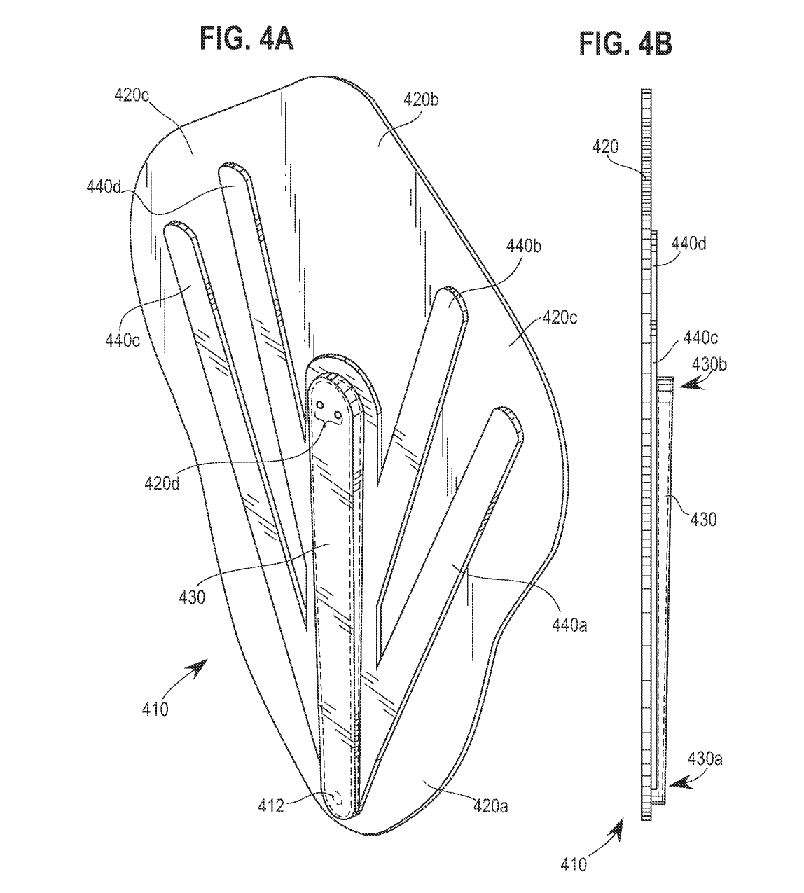

The taper illustrated for spine 130 is a tapering of the width of the spine 130 so that the width of the spine 130 at the rear portion 130b thereof (see dimension B) is greater than the width of the spine 130 at the front portion 130a thereof (see dimension A). It should be appreciated, however, that in alternate embodiments the tapering could be a tapering of the height of the spine 130 at the forward portion 130a thereof either instead of, or in addition to, the tapering of the width of the forward portion 130a of spine 130. Thus, in some forms, the spine may taper in width toward the forward portion 130a as illustrated in FIGS. 1A-E; while in other forms, the spine may taper in height only toward the forward portion 130a; while in still other forms, the spine may taper in both width and height toward the forward portion 130a of spine 130 (as will be discussed further below with respect to FIGS. 4A-B).

Turning back to the embodiment illustrated in FIGS. 1A-E, once the spine 130 is molded, it is then overmolded with an FDA grade PVC material (or other material as discussed above) that forms the flexible outer body 120 of the surgical aid 110. In a preferred form, the spine 130 is positioned along the central axis of the outer body 120 and along the longitudinal axis of the surgical aid 110. More particularly, the forward portion 130a of spine 130 is positioned in or near the forward portion 120a of outer body 120 so that both forward portions 120a, 130a are removed from the wound before the remainder of the body and spine.

Although the illustrated embodiment shows the spine 130 as a separate piece molded into the outer body 120, it should be understood that in alternate forms of the surgical aid 110 the spine 130 could be integrally formed from the same material making-up the rest of the flexible body 120 if desired. The rigidity of the spine in such an embodiment would come from the greater thickness of the material at the spine location as compared to the remainder of the outer body 120. In other forms, the rigidity of the spine 130 may not be an issue or a necessary consideration and, thus, a body and spine of uniform thickness may be used. Similarly, it should be understood that the flexible body 120 could itself be tapered from rear to front (or rearward portion to forward portion) to further make the surgical aid 110 easier to remove from the portion of the wound that remains open when the surgical aid 110 is to be removed from the patient. For example, both the forward portion of the spine 130a and forward portion 120a of the flexible body 120 could be formed with a continuing taper (meaning the taper of the outer body 120 could be configured to follow, continue or track the taper of the spine 130) so that the forward portions 120a, 130a are more easily directed to and pulled through the remaining opening of the wound once it has been partially or mostly closed. This configuration may even allow for more of the wound to be closed so that there is less of a wound opening that needs to be closed after removal of the surgical aid 110. As with the spine, the tapering of the body 120 could be in width, height, or both width and height.

In one form, the surgical aid 110 will further include ribs extending from the spine 130 toward peripheral regions 120c of the flexible body 120. In the form illustrated in FIGS. 1A-E, the body 120 includes a plurality of ribs 140a-d extending out from opposite sides of the spine 130 toward the peripheral portions 120c of the body 120 to support the peripheral portions 120c of the body 120 when the body is in the expanded position (as illustrated in FIGS. 1A-E) so that the medical device or surgical aid 110 can be used to retain and/or protect patient anatomy while the wound is being closed. More particularly, the ribs 140a-d help bias or keep the flexible body 120 in the elongated position so that it more easily can be positioned over anatomy in an open wound without having the peripheral edges of body 120 folding under or over the remaining portions of body 120 and not properly expanding the body 120 over the greatest surface area to ensure that the surgical aid 110 retains the largest portion of the patient's anatomy possible and/or protects the largest possible portion of the patient's anatomy from scrapes, nicks, punctures, tears or cuts while the wound is being closed (e.g., sutured, stapled, etc.). Together with the spine 130, the ribs 140a-d further give weight to the surgical aid 110 and help anchor itself and the anatomy it is intended to retain and/or protect in the patient's body cavity or wound.

In the form illustrated, the ribs 140a-d are sweptback and extend from the spine at small angles. For example, in one form, ribs 140a-d extend from the spine at an angle no more than thirty-three degrees to make the medical device easier to remove from the wound after a portion of the wound has been closed. More particularly, this swept back angle causes the ribs 140a-d to be longer and, thus, more easy to collapse when desired (e.g., when removing the aid 110 from the patient's body after the wound has been partially closed over the aid 110). In the embodiment illustrated, the plurality of ribs have lengths of between fifty-five and one hundred twenty percent (55%-120%) of the length of the spine 130 so that the ribs form elongated ribs that can be more easily moved toward the spine 130 when the body 120 is moved into the collapsed position and so that the medical device 110 can be more easily removed from the wound after a portion of the wound has been closed.

In the form illustrated, the sweptback ribs 140a-d extend from the spine at an angle between ten and thirty-three degrees and, more particularly, the right and left forward ribs 140a, 140c, respectively, form a first set of ribs that extend from opposite sides of the forward portion 130a of spine 130 at a first angle and the right and left rear ribs 140b, 140d, respectively, form a second set of ribs that extend from opposite sides of the rearward portion 130b of spine 130 at a second angle. In the form illustrated, the first and second angles are different from one another and in a preferred form, the first and second sets of ribs will extend out from the spine 130 at angles that diverge from one another so that the distal ends of the ribs extending out from each side of the spine 130 will cover a wider or greater area of the peripheral portions 120c of body 120 and, thereby, provide support for a greater portion of the body 120. In the embodiment shown, the first set of ribs 140a, 140c extend from opposite sides of the forward portion of the spine 130 at an angle between nineteen and twenty-two degrees (19°-22°) and the second set of ribs 140b, 140d extend from opposite sides of the rearward portion of the spine 130 at an angle between fifteen and eighteen degrees (15°-18°) so that the ribs are not parallel with one another and support a larger area of the peripheral portions of the body when in the expanded position while allowing the body to be moved more easily into the collapsed position and for the medical device to be more easily removed from the wound after a portion of the wound has been closed over the aid 110.

It should be understood, however, that in alternate embodiments it may be desired to have the ribs extend out at first and second angles that are generally equal to one another so that the ribs extending from each side of the spine 130 parallel each other. In yet other forms, it may be desired to have the ribs extending from each side of the spine 130 to be of different angles but converging with one another to either meet one another at a specific point located at the peripheral portions 120c of body 120 or at least approach one another at their respective distal ends located at the peripheral portions 120c of the body 120. Similarly, although tapering of the spine 130 and/or body 120 have been discussed above, it should be understood that the ribs 140a-d, like the body 120 and spine 130, have dimensions of length, width and height, and that the ribs could be tapered as well if desired in either height and/or width from the spine toward the distal end of each rib, if desired, in order to make the ribs more flexible and/or easy to contract into the contracted position when removing the aid 110 from the patient's body.

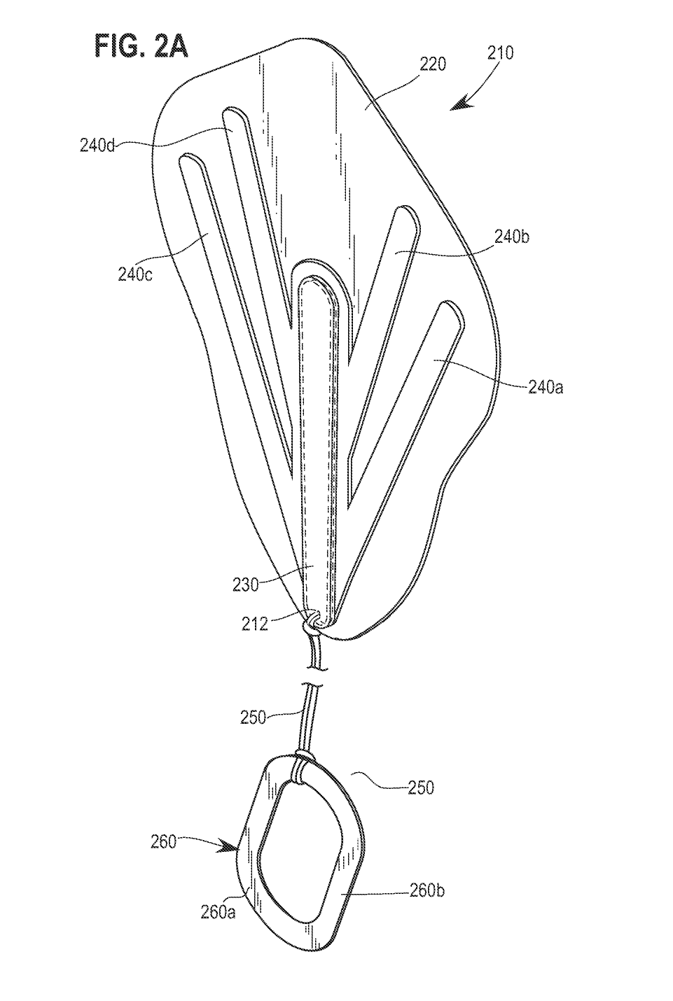

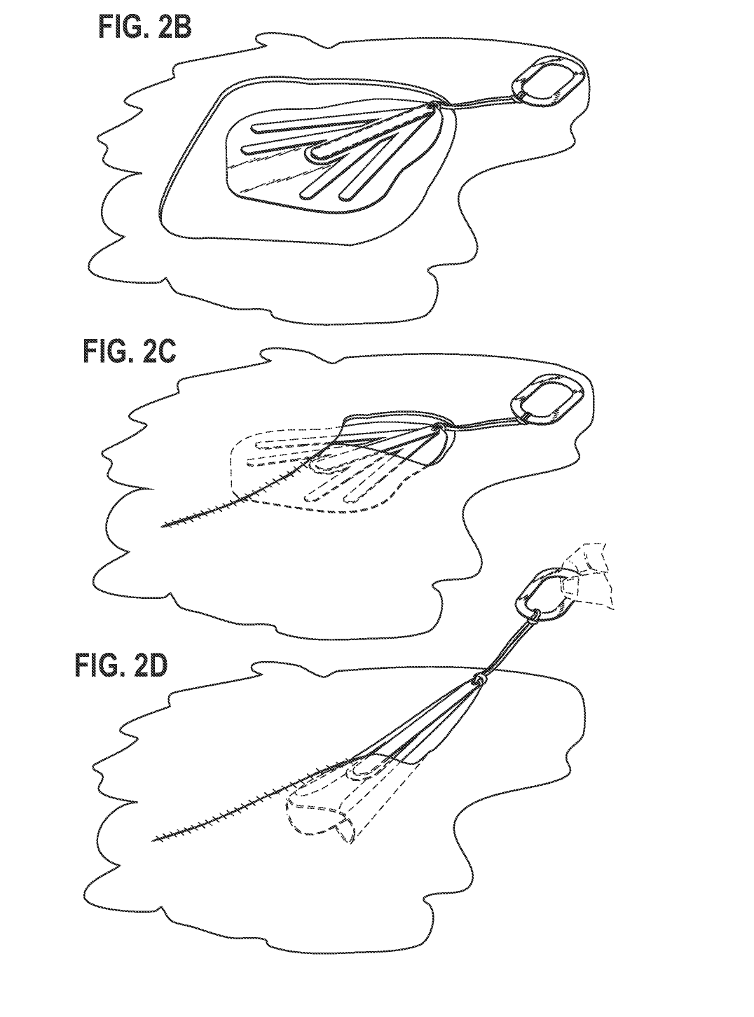

In the form illustrated in FIG. 1A-E, the forward portions 120a, 130a of body 120 and spine 130 define an opening 112 in the front of the medical device or aid 110. The opening 112 may be used to connect a pull cord or handle (not shown) to the aid 110 so that the device can be more easily removed from the patient's body without requiring use of other medical devices such as medical clamps. Such an embodiment will be discussed further now with respect to FIG. 2A.

Turning now to FIGS. 2A-D, there is illustrated an alternate embodiment of a surgical aid or device that includes a pull cord. For convenience, items that are similar to those discussed above with respect to FIGS. 1A-E will use similar two-digit reference numerals, however, with the addition of the prefix “2” simply to distinguish one embodiment from another. In FIG. 2A, the aid 210 includes a flexible body 220, central spine 230 and ribs 240a-d. The forward portions 220a, 230a of body 220 and spine 230, respectively, define an opening 212 through which pull cord 250 is disposed. The pull cord 250 connects to the body 220 and spine 230 in this manner and may be made of any acceptable material, such as string, twine, braided silk, leather, metal wire or cabling (covered or uncovered), plastic, etc. This pull cord 220 provides a handle by which the aid 210 can be grasped by a physician, resident, nurse, aid or other medical personnel to remove the device 210 once the majority of a wound opening has been closed. In the form illustrated, a bight is formed with the pull cord which is then looped or passed through itself to form a cow hitch (aka a lark's head, etc.) to connect the pull cord 220 and the flexible body 220. This hitch knot allows the pull cord 220 to be used to pull the body 220 from the wound even if the ultimate knot connecting the ends of the pull cord 220 together comes undone or if the pull cord 220 breaks or is cut anywhere along the remainder of the loop forming the pull cord 220.

In the form illustrated in FIG. 2A, the aid 210 further includes an additional handle or grip 260 connected to the pull cord 250 which may be grasped by medical personnel to make the removal of the aid 210 even easier to accomplish. In the form illustrated, the handle 260 is connected to the pull cord 250 via a hitch knot, such as another cow hitch, which allows the handle 260 and pull cord 250 to continue to be used to remove the body 220 from a partially closed wound even if the pull cord is cut or breaks somewhere along the remainder of the pull cord 250. The handle 260 preferably has a generally oval or oblong shape so that it can be grasped more easily by a wide range of hand sizes. For example, the narrower end 260a may be grasped and pulled by a smaller hand; whereas, the wider side 260b may be grasped and pulled by a larger hand. Furthermore, in the embodiment shown, the handle 260 is made of a rigid plastic and the pull cord 250 is made of string that is looped through the central opening of the handle 260 and opening 212 of the body 220 and spine 230. In the form illustrated, the retainer handle 260 has an overall height of ninety thousandths of an inch (0.090″), width of two and five hundred thousandths inches (2.500″), and a length of two inches (2.000″), with a central opening that is two inches (2.000″) wide by one and five hundred thousandths inches (1.500″) long. By connecting the pull cord 250 through the body 220 and spine 230, it reinforces the strength of the connection between these components and allows the product to be provided with varying lengths of pull cord 250 to accommodate different sizes of patients, if desired. For example, a longer pull cord 250 may be used with larger sizes of the aid 210 for larger patients. Whereas, pull cords of smaller lengths may be used with smaller sizes of aids 210 for smaller patients. In other forms, a common size pull cord 250 may be used for all sizes of the aid 210.

Thus, the body 220 and spine 230 may be positioned within the wound to retain and/or protect patient anatomy, while the handle is allowed to be placed outside of the wound opening and the patient's body so that it stands out as a reminder to medical personnel that the item has to be removed prior to fully closing the wound. In a preferred form, the handle 260 is made of a first color and the body 220 is made of a second color different from the first color. The first color of the handle 260 may be made brighter than the second color of the body 220 in order to make the handle stick out further or be more visible during medical procedures to reduce the risk that the medical device 210 will inadvertently be left inside a patient when the medical procedure has been concluded. It should be understood that numerous different colors may be used to accomplish this task and that in some forms, both the body 220 and handle 260 may be made of the same color, such as a bright color that attracts the eye to reduce the risk that either the body 220 or handle 250 will be left inside the patient at the conclusion of the medical procedure.

FIGS. 2B-D illustrate how the surgical aid 210 can be positioned within an open wound of a patient to retain and/or protect patient anatomy (FIG. 2B), how the wound can be closed over the surgical aid 210 (FIG. 2C) and how the surgical aid can be pulled from the patient's body after a majority of the wound has been closed by causing the body to move from the expanded position to the retracted portion to fit through the smaller remaining wound opening (FIG. 2D). As discussed above, the taper of the spine 230 and any other components of the aid 210 (e.g., body 220, ribs 240a-d) helps make it easier to guide the forward portions of the aid 210 to and through the remaining wound opening (which is now smaller than it was at the start of the procedure due to the wound being closed over the aid 210). Tests conducted on the aid 210 illustrated in FIGS. 2A-D indicate that an aid made in accordance with this new design requires approximately twenty six percent (26%) less pull force than conventional viscera retainers having spines and ribs. Specifically, the following data was gathered in which the aid 210 was pulled through a one and a quarter inch (1.250″) opening in a material meant to simulate a wound opening of a patient:

| | Conventional | New Retainer | Percentage Pull |
|---|---|---|---|
| | Retainer | Design | Force Reduced |
| Pull | (Newtons) | (Newtons) | (Rounded) |
| 1 | 54 | 42 | 22% |
| 2 | 52 | 40 | 23% |
| 3 | 51 | 38 | 25% |
| 4 | 49 | 37 | 24% |
| 5 | 48 | 36 | 25% |
| 6 | 47 | 34 | 28% |
| 7 | 45 | 33 | 27% |
| 8 | 43 | 32 | 29% |
| 9 | 42 | 30 | 29% |
| 10 | 39 | 27 | 31% |
| Avg. | 47 | 34.9 | 26% |

Thus, the pull force required for removing the aid 210 is reduced on average by approximately twenty six percent (26%) over conventional viscera retainers with spines and ribs, and on individual trials the pull force is reduced by approximately twenty two percent (22%) to thirty one percent (31%).

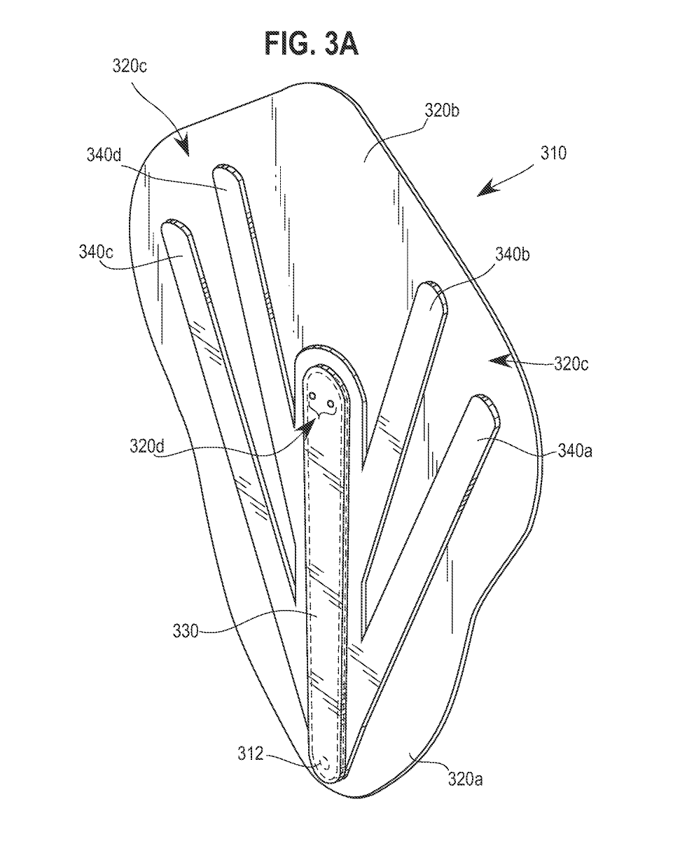

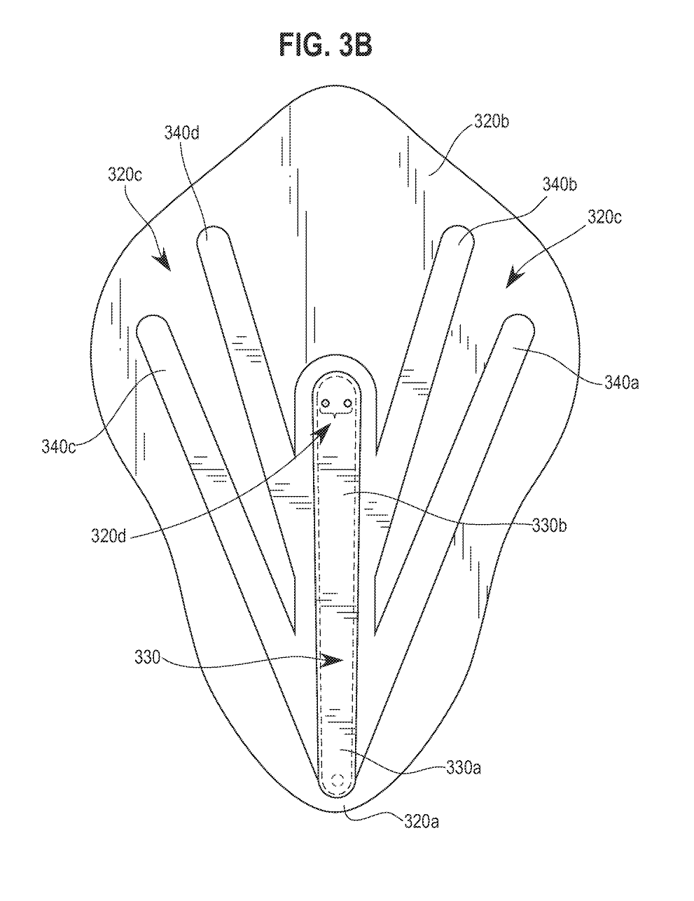

FIGS. 3A-B illustrate an alternate exemplary embodiment of a surgical aid or device in accordance with the invention. In keeping with the above practice and for convenience, items that are similar to those discussed above with respect to FIGS. 1A-2D will use a similar two-digit reference numeral but adding the prefix “3” to distinguish one embodiment from the others. In the form illustrated in FIGS. 3A-B, the surgical aid 310 includes a body 320, spine 330 and ribs 340a-d, with the spine 330 tapering in width from the rearward portion 320b toward the forward portion 320a. In addition, however, the body 320 further defines a plurality of small or minor openings 320d, which are used during the molding process to maintain the position of the center spine 330 as the flexible outer body 320 is molded over the spine 330. These openings 320d allow the spine to be maintained in a steady position so that the spine may be uniformly molded into the outer body 320 and remain near to and parallel with the lower surface or bottom of the outer body 320 to keep the profile of the aid 310 to a minimal size so that the aid 310 is more easy to remove from a patient's body once a wound has been partially closed over and around at least a portion of the aid 310. Although not illustrated, additional small openings or holes will be present on the backside of the spine to further assist in the positioning of the spine 330 during the molding or manufacturing of the aid 310. In the form illustrated, six small openings are aligned along the central axis of the aid 310 and spine 330 to accomplish the alignment of the spine 330 for molding purposes.

FIGS. 4A-B illustrate yet another exemplary embodiment of a surgical aid or device in accordance with the invention. In keeping with above practices, items of this embodiment that are similar to those discussed above with respect to FIGS. 1A-3B will use a similar two-digit reference numeral but adding the prefix “4” to distinguish one embodiment from others. In the form shown in FIGS. 4A-B, the surgical aid 410 includes a body 420, spine 430 and ribs 440a-d, however, in this form the spine 430 not only tapers in width (as illustrated in above embodiments), but further tapers in height, as well. Thus, as best shown in FIG. 4B, the height of the spine 430 tapers from a higher or larger rearward portion 430b toward a shorter or smaller forward portion 430a. This additional taper helps make the spine 430 and entire forward portion of the surgical aid 410 easier to position in and pull through the wound opening after a portion of the wound has been closed over the aid 410.

In looking at the medical device or surgical aid 410 in more detail, the device 410 includes a protective flexible medical grade plastic sheath or body 420 which is generally pear-shaped with indented sides and tapered front and rear portions to assist with easy removal of the device from a patient near the end of the wound closure process. In the middle of the device 410 there is a ridged over-molded center spine 430. The center spine component 430 is molded from polystyrene or a similar ridged plastic alternative with radiopaque material in it so it can be seen under X-ray. This ridged or center spine 430 is also tapered in a couple of ways. First, from the width perspective whereby the rear portion 430b of spine 430 is the widest part of the spine 430 and the forward portion 430a is the narrowest width. The second taper relates to the thickness of the spine 430 whereby the rear portion 430b is up to thirty-five percent (35%) thicker than the forward portion 430a of the spine 430.

The device 410 helps to protect the intestine and other viscera region organs to stay in place and out of harm's way from nicks and needle punctures when suturing the tissue membrane back together and is capable of acting as a third hand for medical personnel trying to retain patient anatomy and/or protect it from being damaged during the wound closure process. The conventional equipment of today (e.g., rigid metal retractors, spatulas, etc.) that is available for retaining and/or protecting patient anatomy either are required to be held by assistants and removed early in the closure procedure or they consist of the ribbed and ribless retainers discussed above in the Background of the Invention and have the problems mentioned therein.

In the form illustrated in FIGS. 4A-B, the spine also has one hole 412 through the thickness of the spine 430 towards the front 430a of the ridged spine 430. In a preferred form, the hole is fifteen hundredths of an inch plus or minus five hundredths of an inch (0.15″±0.05″) in diameter and the orientation is in the center of the radius at the forward portion 430a of spine 430.

Once the ridged spine 430 is molded, it will be over-molded into the body of the retainer 420. The ridged spine 430, when put into the retainer body 420 for over-molding, will have the following orientation: the narrower, thinner portion 430a of the ridged spine 430 will be placed towards the front of the pear-shaped retainer body 420 so that the hole end 430a of the ridged spine 430 encapsulated in the retainer body 420 is at the front portion of the device (see FIGS. 4A-B).

It is contemplated that there will be four (4) sizes of aids made in accordance with the invention disclosed herein, including sizes: small, medium, large, and extra-large. In preferred forms, the widest part of the retainer body 420 will be approximately eight inches (8″) wide with a length that is approximately twelve inches (12″) long, creating a length to width ratio of one and one half to one (1.5:1). This may vary a bit from size to size, but these general dimensions and ratio will attempt to be followed in view of how easy they make the device to install and remove from a patient's wound opening.

The retainer body 420 is made from an FDA grade flexible PVC compound to produce and is green in color. Other flexible plastics/silicones with the same flexibility, density, and weight characteristics can be alternative materials for the retainer body 420 so long as they are also FDA medical/surgical grade materials.

In the form illustrated the molded retainer body 420 preferably has a base thickness or height of six hundredths of an inch plus or minus two hundredths (0.06″±0.02″) and includes two thicker rib portions 440a, 440b and 440c, 440d, respectively, on each side of the ridged over-molded center spine 430. These ribbed sections add thirty-five thousandths of an inch plus or minus five thousandths of an inch (0.035″±0.005″) of thickness to the base thickness, making the total thickness at the rib sections ninety-five thousandths of an inch plus or minus twenty-five thousandths of an inch (0.095″±0.025) thick (or high). These thicker sections 440a-d add some rigidity to the device 410 so that it will lie flat in the abdominal cavity and protect the viscera organs while the closure procedure is performed, but the swept back nature of the ribs as well as the taper of the spine 130 make the device easier to remove from the patient near the end of the wound closure process.

Specific dimensions for exemplary small, medium, large and extra-large devices in accordance with the invention may be as follows. For a small version, the product 410 may have a flexible body 420 width of approximately five and six hundred thousandths inches (5.600″) by a body 420 length of eight and five hundred thousandths inches (8.500″), with a body 420 height of sixty thousandths of an inch (0.060″), a rib portion height of ninety-five thousandths of an inch (0.095″) and a center spine portion height of two hundred thousandths of an inch (0.200″) (which includes the actual height of spine 430 and the height of the overmolded portion of outer body 420 that covers the spine 430. The angle the first rib set 440a, 440c extends from the spine is twenty degrees (20°) and has a leading edge of approximately five and four hundred thousandths inches (5.400″) long. This gives the ribs 440a, 440c a spread of four and five hundred thousandths inches (4.500″) at the outer edge of the distal ends thereof. The angle the second rib set 440b, 440d extends from the spine is seventeen degrees) (17° and the leading edge of each rib 440b, 440d is approximately three and five hundred thousandths inches (3.500″) long. This gives the ribs 440b, 440d a spread of three and one hundred fifty thousandths inches (3.150″) at the outer edge of the distal ends thereof.

A medium version may have a flexible body 420 width of approximately six inches (6.000″) by a body 420 length of nine inches (9.000″), with a body height of the same dimensions as the small version above. Each rib of the first rib set 440a, 440c extends from the spine at an angle of twenty-one degrees (21°) and each has a leading edge of five and eight hundred seventy thousandths inches (5.870″) long. This gives the ribs 440a, 440c a spread of four and nine hundred thousandths inches (4.900″) at the outer edge of the distal ends thereof. The angle the second rib set 440b, 440d extends from the spine is seventeen degrees (17°) and the leading edge of each rib 440b, 440d is approximately four inches (4.000″) long. This gives the ribs 440b, 440d a spread of three and four hundred thousandths inches (3.400″) at the outer edge of the distal ends thereof.

A large version may have a flexible body 420 width of approximately six and five hundred thousandths inches (6.500″) by a body 420 length of nine and five hundred thousandths inches (9.500″), with a body height of the same dimensions as the small version above. Each rib of the first rib set 440a, 440c extends from the spine at an angle of twenty-one degrees (21°) and each has a leading edge of six and four hundred thousandths inches (6.400″) long. This gives the ribs 440a, 440c a spread of five and two hundred eighty thousandths inches (5.280″) at the outer edge of the distal ends thereof. The angle the second rib set 440b, 440d extends from the spine 430 is sixteen degrees (16°) and the leading edge of each rib 440b, 440d is approximately four and six hundred thousandths inches (4.600″) long. This gives the ribs 440b, 440d a spread of three and six hundred fifty thousandths inches (3.650″) at the outer edge of the distal ends thereof.

An extra-large version may have a flexible body 420 width of approximately eight inches (8.000″) by a body 420 length of approximately twelve inches (12.000″), with a body height of the same dimensions as the small version above. The angle the first rib set 440a, 440c extend from the spine 430 is twenty-one degrees plus or minus two degrees (21°±2°) and each having a leading edge of five to eight inches (5″-8″) long. The angle the second rib set 440b, 440d extend from the spine 430 is seventeen degrees plus or minus two degrees (17°±2°) and each having a leading edge of three to six inches (3″-6″) long. In an alternate form, the extra-large version may have a width of eight inches (8.000″) and a body length of ten and five hundred thousandths inches (10.5″), which similarly keeps the width to length ratio to a desired 1:1.5 ratio.

It should be understood, however, that these dimensions are merely exemplary and that alternate embodiments may be provided with varying dimensions but still remaining in accordance with the invention contemplated herein. For example, a longer, narrower version of the surgical aid may be provided, a shorter, wider version may be provided, etc. In other forms any length and width or other dimension ranging between all of the examples described above may be utilized (e.g., a width of five to eight inches (5″-8″), a length of eight to twelve inches (8″-12″), etc.).

As mentioned above, the device 410 is inserted into the wound to act as a protective sheath for the viscera organs from punctures or cuts while closing (e.g., suturing, etc.) the wound. This ridged over-molded spine 430 has a molded hole 412 in the forward end 430a as mentioned above whereby a pull cord may be attached to the device 410. In one form, the opposite end of the pull cord includes a bright green oblong circular handle which remains outside of the patient throughout the surgical procedure to be used to pull and extract the device 410 out of the patient when the wound closure procedure is almost complete. Once the device 410 is pulled through the remaining opening, the remaining wound opening is closed.

The device 410 is primarily, but not exclusively, used for viscera abdominal region wound closures. It should be understood, however, that in alternate embodiments, different sizes of the device 410 may be provided to retain and/or protect other portions of a patient's anatomy as desired.

In view of the above, it should be understood that many methods are also disclosed herein including but not limited to methods of manufacturing and methods of using a surgical aid like the one disclosed herein. For example, disclosed herein is a method of manufacturing a medical device for assisting in a wound closure process of a medical procedure that includes providing a tapered spine member and overmolding the tapered spine member to form a flexible body that is moveable between an expanded position wherein peripheral portions of the body are spaced apart from the spine and a retracted position wherein the peripheral portions of the body are positioned closer to the spine so that the body and spine can be removed from a smaller wound opening than would otherwise be required if the body was in the expanded position. The tapered spine being aligned along a central axis of the body with the tapered end of the spine positioned near a forward portion of the body to make the forward portion of the body easier to remove through the smaller wound opening. In one form, the spine member has first and second ends and dimensions of length, width and height, and the method further includes providing a tapered spine member that includes tapering at least one of the spine width and height of the first end of the spine member so that the first end of the spine member is narrower or smaller than the second end of the spine member and can more easily fit through the remaining wound opening after the wound has be closed over or around at least a portion of the medical device.

The method of overmolding the tapered spine may further include molding a plurality of ribs into the body that extend from the spine member toward peripheral portions of the body to provide support to the peripheral portions of the body. The process of molding the plurality of ribs may include molding sweptback ribs that extend from the spine member at angles between ten and thirty-three degrees (10°-33°) so that the ribs allow the body to more easily moved between the expanded and retracted positions. In one form, the spine member has forward and rearward portions and dimensions of length, width and height, and molding the plurality of ribs comprises molding a first set of ribs that extend from opposite sides of the forward portion of the spine at a first angle with respect to the spine and molding a second set of ribs that extend from opposite sides of the rearward portion of the spine at a second angle with respect to the spine with the second angle being different than the first so that the ribs support a larger portion of the peripheral portions of the body while still allowing the medical device to be easily removed from the patient as the wound closure process nears an end.

In another form, molding the first set of ribs may include molding the first set of ribs to extend from opposite sides of the forward portion of the spine at an angle between nineteen and twenty-two degrees (19°-22°) and molding the second set of ribs to extend from opposite sides of the rearward portion of the spine at an angle between fifteen and eighteen degrees (15°-18°) so that the ribs are not parallel with one another and support a larger portion of the peripheral portions of the body while still allowing the medical device to be easily removed from the patient as the wound closure process nears an end.

In yet another form, the method involves molding ribs of specific length with respect to the length of the spine so that the ribs are more easily collapsible to allow the surgical aid to be removed from a patient more easily. In one form, the spine member has first and second ends and dimensions of length, width and height, and molding the plurality of ribs comprises molding ribs that have a length of between fifty-five and one hundred twenty percent (55%-125%) of the length of the spine so that the ribs allow the body to more easily collapse and, therefore, more easily move between the expanded position and the retracted or collapsed position.

Thus, despite the longstanding problems associated with the Glassman viscera retainer and the fact the only solution suggested and offered to-date was to remove the retainer ribs completely, there is disclosed herein a new, novel and improved surgical aid/medical device that utilizes a unique structure with ribs to help retain patient anatomy and/or protect the patient from scrapes, nicks, punctures, tears or cuts during wound closure processes, but still provide anchorage and retentive power and yet be extracted or removed easily towards the end of the wound closure process.

What is claimed is:

1. A medical device for assisting in a wound closure process of a medical procedure comprising a body made of a flexible material and having a spine located along a central axis of the body, the body being moveable between an expanded position wherein peripheral portions of the body are spaced apart from the spine so that the body and spine can be positioned within a wound to retain or protect patient anatomy and a retracted position wherein the peripheral portions of the body are positioned closer to the spine so that the body and spine can be removed from the wound after a portion of the wound has been closed, the spine further being tapered from a rearward portion of the spine toward a forward portion of the spine with the forward portion of the spine being positioned at or near a forward portion of the body to further assist in removal of the body and spine from the wound after a portion of the wound has been closed.

2. The medical device of claim 1 wherein the body further comprises a plurality of ribs extending out from the spine toward the peripheral portions of the body to support the peripheral portions of the body when the body is in the expanded position so that the medical device can be used to retain or protect patient anatomy while the wound is being closed.

3. The medical device of claim 2 wherein the forward portions of the spine and body define an opening and the medical device further comprises a pull cord connected to the opening defined by the forward portions of the spine and body which may be used to remove the medical device from the wound after a portion of the wound has been closed.

4. The medical device of claim 3 wherein the spine is a separate rigid component molded into the flexible body and the medical device further comprises a handle connected to the pull cord for assisting in removing the medical device from the wound after a portion of the wound has been closed.

5. The medical device of claim 4 wherein the handle is made of a first color and the body is made of a second color different than the first color and the handle color is brighter than the body color to reduce the risk that the medical device will inadvertently be left inside a patient when the medical procedure has been concluded.

6. The medical device of claim 1 wherein the spine has dimensions of length, width and height and the taper of the spine comprises a taper of at least one of the spine width and height.

7. The medical device of claim 2 wherein the plurality of ribs are sweptback and extend from the spine at an angle no more than thirty-three degrees to make the medical device easier to remove from the wound after a portion of the wound has been closed.

8. The medical device of claim 2 wherein the plurality of ribs are sweptback and extend from the spine at an angle between ten and thirty-three degrees to make the medical device easier to remove from the wound after a portion of the wound has been closed.

9. The medical device of claim 8 wherein the plurality of ribs comprises a first set of ribs extending from opposite sides of the forward portion of the spine at a first angle with respect to the spine and a second set of ribs extending from opposite sides of the rearward portion of the spine at a second angle with respect to the spine with the second angle being different than the first.

10. The medical device of claim 9 wherein the ribs on each side of the spine extend out from the spine at divergent angles to one another to cause the ribs to support a larger area of the peripheral portions of the body when in the expanded position while allowing the medical device to be more easily removed from the wound after a portion of the wound has been closed.

11. The medical device of claim 10 wherein the first set of ribs extend from opposite sides of the forward portion of the spine at an angle between nineteen and twenty-two degrees and the second set of ribs extend from opposite sides of the rearward portion of the spine at an angle between fifteen and eighteen degrees so that the ribs are not parallel with one another and support a larger area of the peripheral portions of the body when in the expanded position while allowing the body to be moved more easily into the collapsed position and for the medical device to be more easily removed from the wound after a portion of the wound has been closed.

12. The medical device of claim 2 wherein the spine and ribs each have dimensions of length, width and height, and the plurality of ribs have lengths of between fifty-five and one hundred twenty percent of the length of the spine so that the ribs are elongated ribs that can be more easily moved toward the spine when the body is moved into the collapsed position and so that the medical device can be more easily removed from the wound after a portion of the wound has been closed.

13. The medical device of claim 2 wherein the spine and ribs each have dimensions of length, width and height, and the plurality of ribs comprise tapered ribs which taper from the spine toward a distal end of each rib, with the taper of the spine and ribs being a taper of at least one of the spine and rib width and height.

14. The medical device of claim 2 wherein the spine is made of a radiopaque material so that the medical device remains visible with use of radiation emitting medical equipment.

15. A method of manufacturing a medical device for assisting in a wound closure process of a medical procedure comprising:
providing a tapered spine member; and
overmolding the tapered spine member to form a flexible body that is moveable between an expanded position wherein peripheral portions of the body are spaced apart from the spine and a retracted position wherein the peripheral portions of the body are positioned closer to the spine so that the body and spine can be removed from a smaller wound opening than would otherwise be required if the body was in the expanded position, the tapered spine being aligned along a central axis of the body with the tapered end of the spine positioned near a forward portion of the body to make the forward portion of the body more easy to remove through the smaller wound opening.

16. The method of claim 15 wherein the spine member has first and second ends and dimensions of length, width and height, and providing a tapered spine member comprises tapering at least one of the spine width and height of the first end of the spine member so that the first end of the spine member is narrower or smaller than the second end of the spine member.

17. The method of claim 15 wherein overmolding the tapered spine comprises molding a plurality of ribs into the body that extend from the spine member toward the peripheral portions of the body to provide support to the peripheral portions of the body.

18. The method of claim 17 wherein molding the plurality of ribs comprises molding sweptback ribs that extend from the spine member at angles between ten and thirty-three degrees so that the ribs allow the body to more easily moved between the expanded and retracted positions.

19. The method of claim 17 wherein the spine member has forward and rearward portions and dimensions of length, width and height, and molding the plurality of ribs comprises molding a first set of ribs that extend from opposite sides of the forward portion of the spine at a first angle with respect to the spine and molding a second set of ribs that extend from opposite sides of the rearward portion of the spine at a second angle with respect to the spine with the second angle being different than the first so that the ribs support a larger portion of the peripheral portions of the body while still allowing the medical device to be easily removed from the patient as the wound closure process nears an end.

20. The method of claim 19 wherein molding the first set of ribs comprises molding the first set of ribs to extend from opposite sides of the forward portion of the spine at an angle between nineteen and twenty-two degrees and molding the second set of ribs to extend from opposite sides of the rearward portion of the spine at an angle between fifteen and eighteen degrees so that the ribs are not parallel with one another and support a larger portion of the peripheral portions of the body while still allowing the medical device to be easily removed from the patient as the wound closure process nears an end.

21. The method of claim 17 wherein the spine member has first and second ends and dimensions of length, width and height, and molding the plurality of ribs comprises molding ribs that have a length of between fifty-five and one hundred twenty percent of the length of the spine so that the ribs allow the body to more easily move between the expanded position and the retracted position.

22. A surgical aid for temporary placement in a patient's body cavity to retain at least a portion of the patient's anatomy and/or protect the at least a portion of the patient's anatomy from scrapes, nicks, punctures, tears or cuts while closing the wound, the surgical aid comprising:
a flexible retainer body having forward and rearward portions; and
a ridged spine component that is tapered from a rearward portion to a forward portion and molded into the flexible retainer body, the spine being positioned along a central axis of the flexible body and having the tapered forward portion placed in the forward portion of the retainer body so that the forward portions form an easy to remove tapered first portion of the surgical aid which is the first part of the surgical aid to be extracted from the wound.

23. The surgical aid of claim 22 wherein the spine component has dimensions of width, height and length and the ridged spine component has a width that is not more than sixty hundredths of an inch plus or minus ten hundredths of an inch (0.60″±0.10″) wide at the rearward portion of the spine component and tapers down to no less than twenty hundredths of an inch (0.20″) wide at the forward portion of the spine component, and the spine component is overmolded into the retainer body.

24. The surgical aid of claim 22 whereby the length of the ridged spine component is five and sixty hundredths of an inch plus or minus fifty hundredths of an inch (5.60″±0.50) and the forward portion of the spine component defines an opening of fifteen hundredths of an inch plus or minus five hundredths of an inch (0.15″±0.05″) for receiving a pull cord, the surgical aid further comprising a pull cord connected to the opening defined by the forward portion of the spine component and wherein the spine component is made of a radiopaque material so that the spine component is visible in images taken via electromagnetic radiation imaging.
